SEQUENCE LISTING (19 sequences)

SEQ ID NO: 1 - DNA, 29 bp, Artificial Sequence
  Mycoplasma bacteria Forward primer
ggtgaaatccaggtacgggtgaagacacc

SEQ ID NO: 2 - DNA, 39 bp, Artificial Sequence
  Mycoplasma bacteria Reverse primer
gtcctgatcaatattaagctacagtaaagcttcacgggg

SEQ ID NO: 3 - DNA, 15 bp, Artificial Sequence
  Mycoplasma bacteria Fluorescent probe
cgggacggaaagacc

SEQ ID NO: 4 - DNA, 27 bp, Artificial Sequence
  Mycoplasma bacteria Fluorescent probe
cgttaggcgcaacgggacggaaagacc

SEQ ID NO: 5 - DNA, 20 bp, Artificial Sequence
  Mycoplasma bacteria Forward primer
aaatccaggtacgggtgaag

SEQ ID NO: 6 - DNA, 28 bp, Artificial Sequence
  Mycoplasma bacteria Reverse primer
gtcctgatcaatattaagctacagtaaa

SEQ ID NO: 7 - DNA, 20 bp, Artificial Sequence
  Mycoplasma bacteria Forward primer
aaatccaggtacgggtgaag

SEQ ID NO: 8 - DNA, 28 bp, Artificial Sequence
  Mycoplasma bacteria Reverse primer
gtcctgatcaatattaagctacagtaaa

SEQ ID NO: 9 - DNA, 25 bp, Artificial Sequence
  Mycoplasma bacteria Forward primer
aaatccaggtacgggtgaagacacc

SEQ ID NO: 10 - DNA, 36 bp, Artificial Sequence
  Mycoplasma bacteria Reverse primer
gtcctgatcaatattaagctacagtaaagcttcacg

SEQ ID NO: 11 - DNA, 29 bp, Artificial Sequence
  Mycoplasma bacteria Forward primer
ggtgaaatccaggtacgggtgaagacacc

SEQ ID NO: 12 - DNA, 39 bp, Artificial Sequence; identical to SEQ ID NO: 2
  Mycoplasma bacteria Reverse primer

SEQ ID NO: 13 - DNA, 31 bp, Artificial Sequence
  Mycoplasma bacteria Forward primer
ggtgaaatccaggtacgggtgaagacacccg

SEQ ID NO: 14 - DNA, 50 bp, Artificial Sequence
  Mycoplasma bacteria Reverse primer
catgataatgtcctgatcaatattaagctacagtaaagcttcacggggtc

SEQ ID NO: 15 - DNA, 39 bp, Artificial Sequence
  Mycoplasma bacteria Forward primer
ggtgaaatccaggtacgggtgaagacacccgttaggcgc

SEQ ID NO: 16 - DNA, 77 bp, Artificial Sequence
  Mycoplasma bacteria Reverse primer
gcatcgattgctcctacctattctctacatgataatgtcctgatcaatattaagctacagtaaagcttcacggggtc

SEQ ID NO: 17 - DNA, 31 bp, Artificial Sequence
  Bordetella pertussis Forward primer
atcaagcaccgctttacccgaccttaccgcc

SEQ ID NO: 18 - DNA, 33 bp, Artificial Sequence
  Bordetella pertussis Reverse primer
ttgggagttctggtaggtgtgagcgtaagccca

SEQ ID NO: 19 - DNA, 22 bp, Artificial Sequence
  Bordetella pertussis Fluorescent probe
aatggcaaggccgaacgcttca

(19) United States
(12) Patent Application Publication
(10) Pub. No.: US 2018/0030493 A1
(43) Pub. Date: Feb. 1, 2018

(54) NUCLEIC ACID AMPLIFICATION REACTION METHOD, NUCLEIC ACID AMPLIFICATION REACTION REAGENT, AND METHOD OF USING NUCLEIC ACID AMPLIFICATION REACTION REAGENT

(71) Applicant: Seiko Epson Corporation, Tokyo (JP)

(72) Inventors: Masato HANAMURA, Shiojiri (JP); Kotaro IDEGAMI, Chino (JP); Masayuki UEHARA, Matsumoto (JP)

(21) Appl. No.: 15/652,804

(22) Filed: Jul. 18, 2017

(30) Foreign Application Priority Data
Jul. 29, 2016 (JP) 2016-149491

(51) Int. Cl. C12P 19/34; C12Q 1/68

(52) U.S. Cl. CPC C12P 19/34; C12Q 1/689; C12Q 2600/158

(57) ABSTRACT

A nucleic acid amplification reaction method includes performing thermal cycling for amplifying a nucleic acid for a reaction solution containing a template nucleic acid, a primer, a probe, and a polymerase, wherein in the thermal cycling, the time per cycle of the thermal cycling is 9 seconds or less, the calculated Tm value of the primer is 65° C. or higher and 80° C. or lower, a ΔTm value obtained by subtracting the actually measured Tm value of the primer from the actually measured Tm value of the probe is −11° C. or more and 2° C. or less, the calculated Tm value is a value calculated according to a calculation formula, and the actually measured Tm value is an actually measured value obtained by actual measurement.

### BACKGROUND

### 1. Technical Field

The present invention relates to a nucleic acid amplification reaction method, a nucleic acid amplification reaction reagent, and a method of using a nucleic acid amplification reaction reagent.

### 2. Related Art

In recent years, due to the development of technologies utilizing genes, medical treatments utilizing genes such as gene diagnosis or gene therapy have been drawing attention. In addition, many methods using genes in determination of breed varieties or breed improvement have also been developed in agriculture and livestock industries. As technologies for utilizing genes, technologies such as a PCR (Polymerase Chain Reaction) method are widely used. Nowadays, the PCR method has become an indispensable technology for elucidation of information on biological materials.

The PCR method is a method of amplifying a target nucleic acid by performing thermal cycling for a solution (reaction solution) containing a nucleic acid to be amplified (target nucleic acid) and a reagent. The thermal cycling is a treatment of periodically subjecting the reaction solution to two or more temperature steps. In the PCR method, a method of performing two- or three-step thermal cycling is generally used.

An increase in PCR speed is a necessary technology for reducing the testing time of a genetic test, and has been much expected in the genetic testing industries.

For example, JP-T-2015-520614 (Patent Document 1) discloses a method in which a polymerase is provided at a concentration of at least 0.5 μM and a primer is provided at a concentration of at least 2 μM, and a cycle is completed in a cycle time of less than 20 seconds per cycle.

In the PCR as described above, in order to quantitatively determine the amplified nucleic acid, a probe is used. It is generally said that the Tm value of the probe is preferably higher than the Tm value of the primer by 6° C. to 8° C.

In the case where the Tm value of the probe is lower than the Tm value of the primer, annealing of the primer is likely to occur prior to hybridization of the probe. In such a case, by an elongation reaction by the polymerase, a double strand is formed, and therefore, the probe cannot hybridize. As a result, for example, the probe is not hydrolyzed by the polymerase, and the probe does not emit light in some cases.

As a result of intensive studies, the present inventors found that in the case where the PCR speed is increased, annealing of the primer and hybridization of the probe are different from those in the case where the PCR speed is not increased.

### SUMMARY

An advantage of some aspects of the invention is to provide a nucleic acid amplification reaction method capable of increasing a fluorescence intensity from a probe while increasing the PCR speed. Another advantage of some aspects of the invention is to provide a nucleic acid amplification reaction reagent capable of increasing a fluorescence intensity from a probe while increasing the PCR speed, and a method of using the same.

A nucleic acid amplification reaction method according to an aspect of the invention includes performing thermal cycling for amplifying a nucleic acid for a reaction solution containing a template nucleic acid, a primer, a probe, and a polymerase, wherein in the thermal cycling, the time per cycle of the thermal cycling is 9 seconds or less, the calculated Tm value of the primer is 65° C. or higher and 80° C. or lower, a ΔTm value obtained by subtracting the actually measured Tm value of the primer from the actually measured Tm value of the probe is −11° C. or more and 2° C. or less, the calculated Tm value is a value calculated according to the following formula (1), and the actually measured Tm value is an actually measured value obtained by actual measurement.

Tm=1000 ΔH/(−10.8+ΔS+R×ln(Ct/4))−273.15+16.6 log [Na⁺] (1)

In the formula (1), ΔH represents the sum (kcal/mol) of the nearest neighbor enthalpy changes for hybrids, ΔS represents the sum (cal/mol/K) of the nearest neighbor entropy changes for hybrids, R represents the gas constant (1.987 cal/deg/mol), Ct represents the molar concentration (mol/L) of the primer, and Na⁺ represents the concentration (mol/L) of a monovalent cation contained in the buffer.

According to such a nucleic acid amplification reaction method, while increasing the PCR speed, a fluorescence intensity from the probe can be increased (see the below-mentioned “3. Experimental Examples” for the details).

In the nucleic acid amplification reaction method according to the aspect of the invention, a heating time for an annealing reaction for the primer may be 6 seconds or less.

According to such a nucleic acid amplification reaction method, while increasing the PCR speed, a fluorescence intensity from the probe can be increased (see the below-mentioned “3. Experimental Examples” for the details).

In the nucleic acid amplification reaction method according to the aspect of the invention, the probe may contain an artificial nucleic acid.

According to such a nucleic acid amplification reaction method, while suppressing an increase in the number of bases of the probe, the ΔTm value can be made to fall within the above range.

In the nucleic acid amplification reaction method according to the aspect of the invention, the probe may contain a minor groove binder molecule.

According to such a nucleic acid amplification reaction method, while suppressing an increase in the number of bases of the probe, the ΔTm value can be made to fall within the above range.

In the nucleic acid amplification reaction method according to the aspect of the invention, the ΔTm value may be −5° C. or more and 2° C. or less.

According to such a nucleic acid amplification reaction method, while increasing the PCR speed, a fluorescence intensity from the probe can be increased (see the below-mentioned “3. Experimental Examples” for the details).

In the nucleic acid amplification reaction method according to the aspect of the invention, the reaction solution may contain a divalent cation, and the concentration of the divalent cation contained in the reaction solution may be 2 mM or more and 7.5 mM or less.

According to such a nucleic acid amplification reaction method, while accelerating an elongation reaction by a polymerase and increasing the PCR speed, nonspecific amplification is suppressed, and a decrease in yield of a specific amplification product can be suppressed.

In the nucleic acid amplification reaction method according to the aspect of the invention, the reaction solution may contain MgCl₂, the divalent cation may be derived from MgCl₂, and the concentration of MgCl₂ contained in the reaction solution may be 4 mM or more and 7.5 mM or less.

According to such a nucleic acid amplification reaction method, while accelerating an elongation reaction by a polymerase and increasing the PCR speed, a decrease in yield of a specific amplification product due to an increase in nonspecific amplification because of too much Mg²⁻ can be prevented from occurring.

In the nucleic acid amplification reaction method according to the aspect of the invention, the reaction solution may contain MgSO₄, the divalent cation may be derived from MgSO₄, and the concentration of MgSO₄ contained in the reaction solution may be 2 mM or more and 3 mM or less.

According to such a nucleic acid amplification reaction method, while accelerating an elongation reaction by a polymerase and increasing the PCR speed, a decrease in yield of a specific amplification product due to an increase in nonspecific amplification because of too much Mg²⁻ can be prevented from occurring.

In such a nucleic acid amplification reaction method, an optimal concentration range of the divalent cation for suppressing nonspecific amplification and suppressing a decrease in yield of a specific amplification product while accelerating an elongation reaction and increasing the PCR speed varies depending on the type of the divalent cation.

A nucleic acid amplification reaction reagent according to an aspect of the invention is a nucleic acid amplification reaction reagent for amplifying a nucleic acid, and includes a primer, a probe, a polymerase, and MgCl₂, wherein when the nucleic acid amplification reaction reagent becomes a reaction solution for performing a nucleic acid amplification reaction, the concentration of MgCl₂ contained in the reaction solution is 4 mM or more and 7.5 mM or less, the calculated Tm value of the primer is 65° C. or higher and 80° C. or lower, a ΔTm value obtained by subtracting the actually measured Tm value of the primer from the actually measured Tm value of the probe is −11° C. or more and 2° C. or less, the calculated Tm value is a value calculated according to the following formula (1), and the actually measured Tm value is an actually measured value obtained by actual measurement.

Tm=1000 ΔH/(−10.8+ΔS+R×ln(Ct/4))−273.15+16.6 log [Na⁺] (1)

In the formula (1), ΔH represents the sum (kcal/mol) of the nearest neighbor enthalpy changes for hybrids, ΔS represents the sum (cal/mol/K) of the nearest neighbor entropy changes for hybrids, R represents the gas constant (1.987 cal/deg/mol), Ct represents the molar concentration (mol/L) of the primer, and Na⁺ represents the concentration (mol/L) of a monovalent cation contained in the buffer.

According to such a nucleic acid amplification reaction reagent, while increasing the PCR speed, a fluorescence intensity from the probe can be increased.

A nucleic acid amplification reaction reagent according to an aspect of the invention is a nucleic acid amplification reaction reagent for amplifying a nucleic acid, and includes a primer, a probe, a polymerase, and MgSO₄, wherein when the nucleic acid amplification reaction reagent becomes a reaction solution for performing a nucleic acid amplification reaction, the concentration of MgSO₄ contained in the reaction solution is 2 mM or more and 3 mM or less, the calculated Tm value of the primer is 65° C. or higher and 80° C. or lower, a ΔTm value obtained by subtracting the actually measured Tm value of the primer from the actually measured Tm value of the probe is −11° C. or more and 2° C. or less, the calculated Tm value is a value calculated according to the following formula (1), and the actually measured Tm value is an actually measured value obtained by actual measurement.

Tm=1000 ΔH/(−10.8+ΔS+R×ln(Ct/4))−273.15+16.6 log [Na⁺] (1)

In the formula (1), ΔH represents the sum (kcal/mol) of the nearest neighbor enthalpy changes for hybrids, ΔS represents the sum (cal/mol/K) of the nearest neighbor entropy changes for hybrids, R represents the gas constant (1.987 cal/deg/mol), Ct represents the molar concentration (mol/L) of the primer, and Na⁺ represents the concentration (mol/L) of a monovalent cation contained in the buffer.

According to such a nucleic acid amplification reaction reagent, while increasing the PCR speed, a fluorescence intensity from the probe can be increased.

A method of using a nucleic acid amplification reaction reagent according to an aspect of the invention is a method of using the nucleic acid amplification reaction reagent according to the aspect of the invention, including preparing the reaction solution by bringing the nucleic acid amplification reaction reagent and a template nucleic acid solution containing a template nucleic acid into contact with each other, and amplifying a nucleic acid by performing thermal cycling in which the time per cycle of the thermal cycling is 9 seconds or less for the reaction solution.

According to such a method of using a nucleic acid amplification reaction reagent, while increasing the PCR speed, a fluorescence intensity from the probe can be increased.

### BRIEF DESCRIPTION OF THE DRAWINGS

The invention will be described with reference to the accompanying drawings, wherein like numbers reference like elements.

FIG. 1 is a graph showing a relationship between a temperature for Taq polymerase and a relative activity efficiency.

FIG. 2 is a flowchart for illustrating a nucleic acid amplification reaction method according to an embodiment.

FIG. 3 is a cross-sectional view schematically showing a thermal cycler for performing thermal cycling for a reaction solution according to an embodiment.

FIG. 4 is a graph showing a relationship between a value obtained by subtracting the Tm value of a primer from the Tm value of a probe and a relative fluorescence intensity.

FIG. 5 is a graph showing a relationship between a PCR reaction time and a fluorescence intensity.

FIG. 6 is a graph showing a relationship between a PCR reaction time and a fluorescence intensity.

### DESCRIPTION OF EXEMPLARY EMBODIMENTS

Hereinafter, preferred embodiments of the invention will be described in detail with reference to the accompanying drawings. Note that the embodiments described below are not intended to unduly limit the content of the invention described in the appended claims. Further, all the configurations described below are not necessarily essential components of the invention.

### 1. NUCLEIC ACID AMPLIFICATION REACTION REAGENT

First, a nucleic acid amplification reaction reagent according to this embodiment will be described. The nucleic acid amplification reaction reagent is a reagent for amplifying a nucleic acid in a nucleic acid amplification reaction (PCR). The nucleic acid amplification reaction reagent may be, for example, in a liquid form or may be in a lyophilized state. For example, the nucleic acid amplification reaction reagent in a lyophilized state is fixed in a container (not shown), and a template nucleic acid solution containing a DNA (deoxyribonucleic acid) or an RNA (ribonucleic acid) is introduced into the container so as to bring the template nucleic acid solution and the nucleic acid amplification reaction reagent into contact with each other. The nucleic acid amplification reaction reagent in a lyophilized state is dissolved in the aqueous component of the template nucleic acid solution and incorporated into the template nucleic acid solution so as to become a reaction solution. Therefore, the reaction solution contains the template nucleic acid and the nucleic acid amplification reaction reagent, and thus serves as a place for allowing a nucleic acid amplification reaction to proceed.

The nucleic acid amplification reaction reagent contains a primer, a polymerase, a probe, dNTP, and a buffer.

### 1.1. Primer

The primer is designed to anneal to a template nucleic acid (template). The “anneal (annealing)” refers to an action (a phenomenon) in which a primer binds to a DNA. The nucleic acid amplification reaction reagent contains a forward primer which anneals to one template nucleic acid having a single-stranded structure (single-stranded DNA) after a template nucleic acid having a double-stranded structure (double-stranded DNA) is denatured, and a reverse primer which anneals to the other single-stranded DNA as the primer. The concentrations of the forward primer and the reverse primer contained in the reaction solution are each, for example, 0.4 μM or more and 6.4 μM or less, preferably 0.8 μM or more and 3.2 μM or less. The concentration of the forward primer and the concentration of the reverse primer contained in the reaction solution may be the same as or different from each other.

The calculated Tm value of the primer (the forward primer and the reverse primer) is 65° C. or higher and 80° C. or lower, preferably 70° C. or higher and 75° C. or lower. According to this, the nucleic acid amplification reaction reagent according to this embodiment can increase the PCR speed (see the below-mentioned “3. Experimental Examples” for the details). The Tm value is an index of the temperature at which a primer anneals to a template nucleic acid, and is a temperature at which 50% of a double-stranded DNA is dissociated into single-stranded DNAs, that is, a melting temperature. If the temperature is not lower than the Tm value, not less than half of the primer anneals to the template nucleic acid. The Tm value of the forward primer and the Tm value of the reverse primer may be the same as or different from each other.

As a calculation method of the Tm value, for example, a nearest neighbor method is exemplified, and the Tm value can be calculated according to the following formula (1). The “calculated Tm value” refers to a Tm value calculated according to the following formula (1).

Tm=1000 ΔH/(−10.8+ΔS+R×ln(Ct/4))−273.15+16.6 log [Na⁺] (1)

In the formula (1), ΔH represents the sum (kcal/mol) of the nearest neighbor enthalpy changes for hybrids, ΔS represents the sum (cal/mol/K) of the nearest neighbor entropy changes for hybrids, R represents the gas constant (1.987 cal/deg/mol), Ct represents the molar concentration (mol/L) of the primer, and Na⁺ represents the concentration (mol/L) of a monovalent cation contained in the buffer.

The Tm value can also be determined by actual measurement. In the case where the Tm value is determined by actual measurement, a given fluorescent substance is bound to a double-stranded DNA formed by the primer and a complementary strand thereto, and a decrease in the emission intensity from the fluorescent substance due to thermal denaturation is plotted against the temperature. A temperature at which a negative primary differential value of this graph reached a peak can be measured as the “actually measured Tm value” (see the below-mentioned “3. Experimental Examples” for the details).

The primer may contain an artificial nucleic acid. According to this, the calculated Tm value of the primer can be made to fall within the above range without increasing the number of bases of the primer. When the number of bases of the primer is increased, nonspecific adsorption occurs or the primers form a primer dimer (double strand), and a target nucleic acid cannot be amplified in some cases. As the primer dimer, there are a self-dimer which forms a double strand in one primer, and a cross-dimer which forms a double strand between the forward primer and the reverse primer.

The “artificial nucleic acid” refers to a nucleic acid molecule which can bind to abase of a DNA or an RNA through a hydrogen bond and is other than natural nucleic acid molecules. Examples of the artificial nucleic acid include a 2′,4′-BNA (2′-0,4′-C-methano-bridged nucleic acid, also known as “LNA (Locked Nucleic Acid)”) in which the oxygen atom at the 2′-position of a ribose ring of a nucleic acid is methylene-crosslinked to the carbon atom at the 4′-position. The chemical formula of the LNA is shown in the following formula (2).

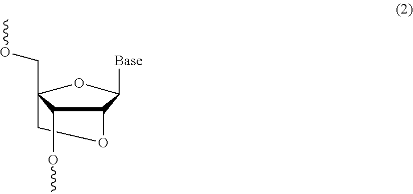

In the formula (2), examples of the base include T (thymine), C (cytosine), G (guanine), and A (adenine), but are not particularly limited. Further, the base may be a base modified by methylation, acetylation, or the like.

The artificial nucleic acid may be an LNA analog obtained by modifying an LNA, and specifically may be 3′-amino-2′,4′-BNA, 2′,4′-BNA^{COC}, or 2′,4′-BNA^{NC} (N-Me). Further, an artificial nucleic acid contained in a modified fluorescent probe may be a PNA (Peptide Nucleic Acid), a GNA (Glycol Nucleic Acid), a TNA (Threose Nucleic Acid), or an analog obtained by modifying such a molecule. The number of artificial nucleic acids contained in the probe is not particularly limited, and one probe may contain a plurality of artificial nucleic acids.

In the case where the Tm value of the primer is increased by increasing the number of bases of the primer, by designing the primer so as to be elongated inside the amplification region (so that the primer is elongated on the 5′-end side of the template nucleic acid), the Tm value can be increased without increasing the amplification region of a nucleic acid by the elongation reaction. According to this, the PCR speed can be increased.

### 1.2. Polymerase

The polymerase is not particularly limited, however, examples thereof include a DNA polymerase. The DNA polymerase polymerizes nucleotides complementary to the bases of a template nucleic acid at the end of the primer annealing to the template nucleic acid having a single-stranded structure (single-stranded DNA). The DNA polymerase is preferably a heat-resistant enzyme or an enzyme for PCR, and there are a large number of commercially available products, for example, Taq polymerase, KOD polymerase, Tfipolymerase, Tthpolymerase, modified forms thereof, and the like, however, a DNA polymerase capable of performing hot start is preferred. As the polymerase, there are a hydrolysis-type polymerase which degrades a probe by hydrolysis such as Taq polymerase, and a non-hydrolysis-type polymerase which does not degrade a probe by hydrolysis such as KOD polymerase. The KOD polymerase is derived from Thermococcus kodakarensis KOD1 and is a DNA polymerase from the genus Thermococcus. The amount of the polymerase contained in the reaction solution is, for example, 0.5 U or more.

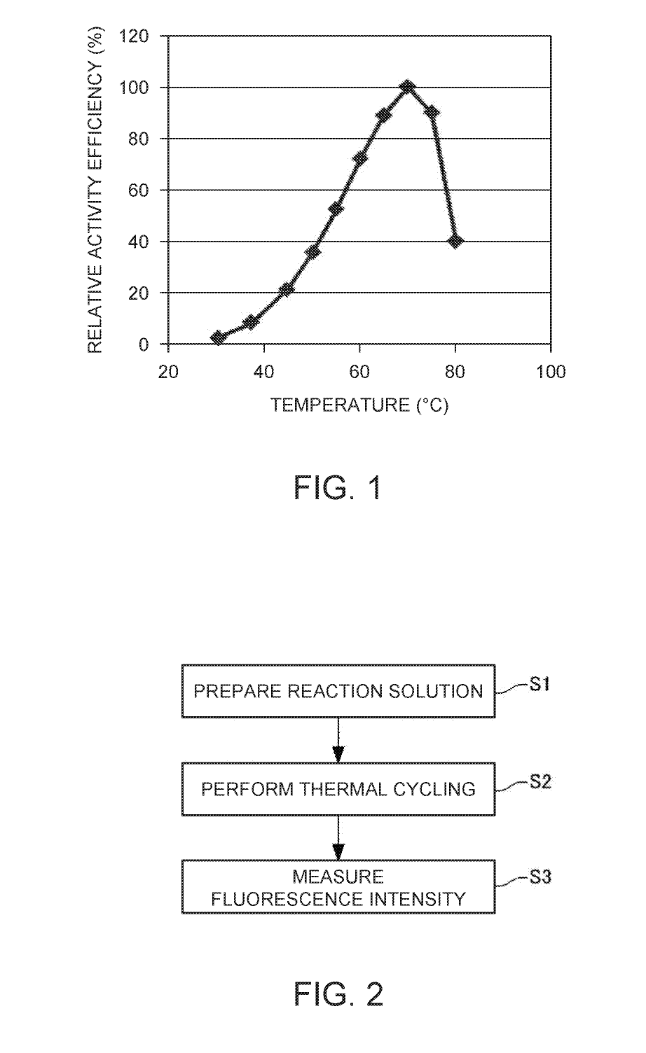

FIG. 1 is a graph showing a relationship between a temperature for Taq polymerase and a relative activity efficiency. The vertical axis represents a relative activity efficiency when the maximum value of the activity efficiency of the polymerase which is reached while changing the temperature is assumed to be 100%. The activity efficiency of the polymerase at each temperature can be obtained by performing a procedure so that dNTP emits light when it is incorporated during the elongation reaction, and measuring the fluorescence intensity (fluorescent brightness) from the dNTP after a predetermined period of time has elapsed. As the fluorescence intensity is higher, the activity efficiency of the polymerase is higher. In the example shown in FIG. 1, the relative activity efficiency of the Taq polymerase reached the maximum when the temperature was around 70° C.

### 1.3. dNTP

The dNTP refers to a mixture of four types of deoxyribonucleotide triphosphates. That is, the dNTP refers to a mixture of dATP, dCTP, dGTP, and dTTP. The DNA polymerase forms a new DNA by joining dATP, dCTP, dGTP, or dTTP to the end of the primer annealing to the template (an elongation reaction). The concentration of the dNTP contained in the reaction solution is, for example, 0.06 mM or more and 0.75 mM or less, preferably 0.125 mM or more and 0.5 mM or less.

### 1.4. Probe

The probe is a fluorescently labeled probe to be used for quantitatively determining the amplification amount of a nucleic acid. The concentration of the probe contained in the reaction solution is 0.5 μM or more and 2.4 μM or less, preferably 0.5 μM or more and 1.8 μM or less.

The probe is, for example, a hydrolysis probe containing a reporter dye and a quencher dye. More specifically, the probe is TaqMan (registered trademark) probe. While the hydrolysis probe hybridizes to a single-stranded DNA to form a double-stranded structure, the light emission of a reporter dye is suppressed by a quencher dye (by a quenching effect) which is in close proximity to the reporter dye. However, when the probe is degraded by the exonuclease activity of the polymerase, the quenching effect is cancelled, and therefore, the reporter dye emits light. By this light emission, the amplification amount of a nucleic acid can be quantitatively determined. The “hybridization” refers to a phenomenon in which a probe binds to a DNA. In the case where a hydrolysis probe is used as the probe, Taq polymerase is used as the polymerase.

The probe may be a non-hydrolysis probe other than the hydrolysis probe. Specifically, the probe may be a Q (Quenching) probe utilizing a fluorescence-quenching phenomenon. The Q probe emits light in a state where it does not hybridize to a single-stranded DNA, and quenches the light when it hybridizes to a single-stranded DNA. By this difference in the emission intensity, the amplification amount of a nucleic acid can be quantitatively determined. In the case where the Q probe is used as the probe, KOD polymerase is used as the polymerase. The elongation reaction rate of KOD polymerase is larger than that of Taq polymerase, and therefore, KOD polymerase can increase the thermal cycling speed.

In the case where the probe is a non-hydrolysis probe, it is not necessary to degrade the probe in the elongation reaction, and therefore, there is no need to provide an amplification region to which the probe anneals between the forward primer and the reverse primer. According to this, it becomes possible to design the amplification region narrower than in a hydrolysis-type system. Since the amplification region becomes narrower, the annealing time can be reduced, and thus, the thermal cycling speed can be increased.

A value (ΔTm value) obtained by subtracting the actually measured Tm value of the primer from the actually measured Tm value of the probe is −11° C. or more and 2° C. or less. That is, in the case where the actually measured Tm value of the primer is 75° C., the actually measured Tm value of the probe is 64° C. or higher and 77° C. or lower. According to this configuration, the nucleic acid amplification reaction reagent according to this embodiment can increase the fluorescence intensity from the probe while increasing the PCR speed (see the below-mentioned “3. Experimental Examples” for the details). The ΔTm value is preferably −5° C. or more and 2° C. or less, more preferably −4.5° C. or more and 1° C. or less.

In the case where the actually measured Tm value of the forward primer and the actually measured Tm value of the reverse primer are different from each other, the ΔTm value is a value obtained by subtracting the average of the actually measured Tm value of the forward primer and the actually measured Tm value of the reverse primer from the actually measured Tm value of the probe.

The probe may contain an artificial nucleic acid. The probe may contain a minor groove binder (MGB) molecule. By containing an artificial nucleic acid or an MGB molecule in the probe, the ΔTm value can be made to fall within the above range while suppressing an increase in the number of bases of the probe (while suppressing an increase in the base length). When the number of bases of the probe is increased, for example, a time for degrading the probe is increased, and therefore, it is sometimes difficult to increase the PCR speed. As the artificial nucleic acid, those listed in “1.1. Primer” can be used.

### 1.5. Buffer

The buffer is, for example, a buffer agent containing a salt. Examples of the salt contained in the buffer include salts such as Tris, HEPES, PIPES, and phosphates. By using such a salt, the pH of the buffer can be adjusted.

The buffer contains a divalent cation. Examples of the divalent cation include Mn²⁺, Co²⁺, and Mg²⁺. In the case where the nucleic acid amplification reaction reagent becomes a reaction solution for performing a nucleic acid amplification reaction, the concentration of the divalent cation contained in the reaction solution is 2 mM or more and 7.5 mM or less. By setting the concentration of the divalent cation to 2 mM or more, the elongation reaction by the polymerase is accelerated, and the PCR speed can be increased (specifically, the time per cycle of the thermal cycling can be reduced to 9 seconds or less). By setting the concentration of the divalent cation to 7.5 mM or less, nonspecific amplification is suppressed, and a decrease in yield of a specific amplification product can be suppressed.

Specifically, the buffer contains a divalent cationic compound, KCl, and Tris. More specifically, the buffer contains MgCl₂, and the divalent cation is derived from MgCl₂. That is, the divalent cation is produced by ionization of MgCl₂. In the case where the divalent cation is derived from MgCl₂, the concentration of Mg²⁺ is attributed to the activity of the polymerase. In the case where the nucleic acid amplification reaction reagent becomes a reaction solution for performing a nucleic acid amplification reaction, the concentration of MgCl₂ contained in the reaction solution is 4 mM or more and 7.5 mM or less, preferably 5 mM or more and mM or less, more preferably 5 mM. By setting the concentration of MgCl₂ to 4 mM or more, the elongation reaction by the polymerase is accelerated, and the PCR speed can be increased. By setting the concentration of MgCl₂ to 7.5 mM or less, nonspecific amplification is suppressed, and a decrease in yield of a specific amplification product can be suppressed. When the nucleic acid amplification reaction reagent is in a lyophilized state, the nucleic acid amplification reaction reagent is in a solid state, and contains MgCl₂, KCl, Tris, and an excipient such as trehalose.

The divalent cationic compound may be derived from MgSO₄. In this case, the buffer contains MgSO₄ in place of MgCl₂, and in the case where the nucleic acid amplification reaction reagent becomes a reaction solution for performing a nucleic acid amplification reaction, the concentration of MgSO₄ contained in the reaction solution is 2 mM or more and 3 mM or less, more preferably 2 mM. By setting the concentration of MgSO₄ to 2 mM or more, the elongation reaction by the polymerase is accelerated, and the PCR speed can be increased. By setting the concentration of MgSO₄ to 3 mM or less, nonspecific amplification is suppressed, and a decrease in yield of a specific amplification product can be suppressed.

### 1.6. Other Components

In the case where an RNA is used as the template nucleic acid, the nucleic acid amplification reaction reagent further contains a reverse transcriptase. As the reverse transcriptase, for example, a reverse transcriptase derived from avian myeloblast virus, Ras-associated virus type 2, mouse Moloney murine leukemia virus, or human immunodefficiency virus type 1 is used.

In the case where the nucleic acid amplification reaction reagent is lyophilized, the nucleic acid amplification reaction reagent (lyophilized reagent) contains a sugar. Examples of the sugar include sucrose, trehalose, raffinose, and melezitose, each of which is a non-reducing sugar, among disaccharides and trisaccharides. Among the disaccharides and trisaccharides, particularly trehalose is preferably used because the function as a cryoprotective agent is high. Trehalose prevents the lyophilized reagent from coming into contact with a water molecule by its strong hydration force, and thus can improve the storage stability of the lyophilized reagent. The lyophilized reagent can be prepared by lyophilizing a mixed reagent solution containing the respective components of the nucleic acid amplification reaction reagent and a sugar. The temperature during lyophilization is, for example, about −80° C.

### 1.7. Using Method

In the method of using the nucleic acid amplification reaction reagent according to this embodiment, a reaction solution is prepared by bringing the nucleic acid amplification reaction reagent and a template nucleic acid solution containing a template nucleic acid into contact with each other, and a nucleic acid is amplified by performing thermal cycling in which the time per cycle of the thermal cycling is 9 seconds or less for the reaction solution.

The nucleic acid amplification reaction reagent according to this embodiment has, for example, the following characteristics.

In the nucleic acid amplification reaction reagent, the calculated Tm value of the primer is 65° C. or higher and 80° C. or lower, and a ΔTm value obtained by subtracting the actually measured Tm value of the primer from the actually measured Tm value of the probe is −11° C. or more and 2° C. or less. Therefore, according to the nucleic acid amplification reaction reagent, while increasing the PCR speed (while reducing the time for PCR), the fluorescence intensity from the probe can be increased, and therefore, the amplification amount of a nucleic acid can be quantitatively determined with high sensitivity (see the below-mentioned “3. Experimental Examples” for the details).

In the nucleic acid amplification reaction reagent, when the nucleic acid amplification reaction reagent becomes a reaction solution for performing a nucleic acid amplification reaction, the concentration of MgCl₂ contained in the reaction solution may be 4 mM or more and 7.5 mM or less. Therefore, according to the nucleic acid amplification reaction reagent, while accelerating an elongation reaction by a polymerase and increasing the PCR speed, a decrease in yield of a specific amplification product due to an increase in nonspecific amplification because of too much Mg²⁺ can be prevented from occurring.

In the nucleic acid amplification reaction reagent, when the nucleic acid amplification reaction reagent becomes a reaction solution for performing a nucleic acid amplification reaction, the concentration of MgSO₄ contained in the reaction solution is 2 mM or more and 3 mM or less. Therefore, according to the nucleic acid amplification reaction reagent, while accelerating an elongation reaction by a polymerase and increasing the PCR speed, a decrease in yield of a specific amplification product due to an increase in nonspecific amplification because of too much Mg²⁺ can be prevented from occurring.

In the nucleic acid amplification reaction reagent, the probe may contain an artificial nucleic acid. Therefore, according to the nucleic acid amplification reaction reagent, while suppressing an increase in the number of bases of the probe, the ΔTm value can be made to fall within the above range.

In the nucleic acid amplification reaction reagent, the probe may contain an MGB molecule. Therefore, according to the nucleic acid amplification reaction reagent, while suppressing an increase in the number of bases of the probe, the ΔTm value can be made to fall within the above range.

In the nucleic acid amplification reaction reagent, the ΔTm value may be −5° C. or more and 2° C. or less. Therefore, according to the nucleic acid amplification reaction reagent, while increasing the PCR speed, the fluorescence intensity from the probe can be further increased (see the below-mentioned “3. Experimental Examples” for the details).

### 2. NUCLEIC ACID AMPLIFICATION REACTION METHOD

Next, the nucleic acid amplification reaction method according to this embodiment will be described with reference to the accompanying drawings. FIG. 2 is a flowchart for illustrating the nucleic acid amplification reaction method according to this embodiment.

First, a reaction solution is prepared by bringing the nucleic acid amplification reaction reagent according to this embodiment and a template nucleic acid solution into contact with each other (Step S1). Specifically, a template nucleic acid solution is introduced using a pipette or the like into a container in which the nucleic acid amplification reaction reagent is placed so as to bring the nucleic acid amplification reaction reagent and the template nucleic acid solution into contact with each other, whereby a reaction solution is prepared. The reaction solution contains, for example, a template nucleic acid, a primer, a probe, a polymerase, dNTP, and a buffer.

The template nucleic acid solution is obtained, for example, as follows. That is, a specimen, for example, a cell derived from an organism such as a human or a bacterium, a virus, or the like is collected using a collecting tool such as a cotton swab, and a template nucleic acid is extracted from the specimen using a known extraction method. Thereafter, a template nucleic acid solution is purified so as to have a predetermined concentration using a known purification method. The solution in the template nucleic acid solution is, for example, water (distilled water or sterile water) or a Tris-EDTA (ethylenediaminetetraacetic acid) (TE) solution.

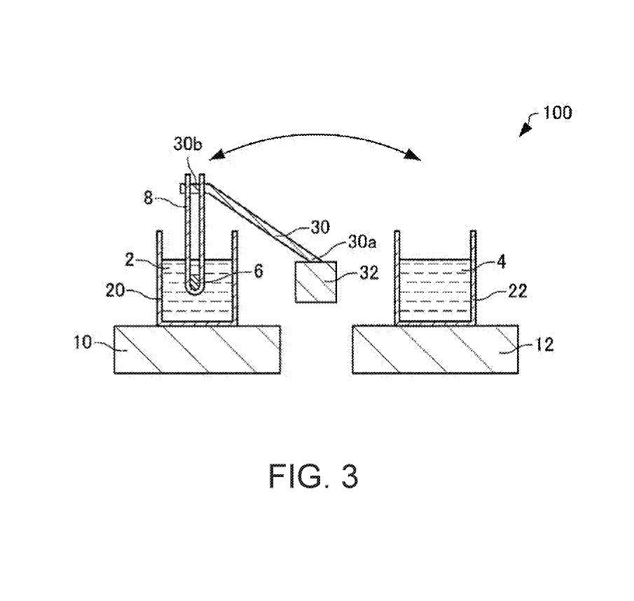

Subsequently, thermal cycling (for PCR) for amplifying a nucleic acid is performed for the reaction solution (Step S2). Here, FIG. 3 is a cross-sectional view schematically showing a thermal cycler 100 for performing thermal cycling for a reaction solution 6 according to this embodiment.

As shown in FIG. 3, the thermal cycler 100 includes a first hot plate 10, a second hot plate 12, a first beaker 20, a second beaker 22, an arm 30, and a fixing section 32.

The first hot plate 10 heats a liquid 2 contained in the first beaker 20 to a first temperature. The first temperature is a temperature suitable for the dissociation (denaturation reaction) of a double-stranded DNA, and is, for example, 85° C. or higher and 105° C. or lower. The liquid 2 is not particularly limited as long as it can be heated to the first temperature by the first hot plate 10, and for example, an aqueous sodium chloride solution and an oil can be exemplified.

The second hot plate 12 heats a liquid 4 contained in the second beaker 22 to a second temperature. The second temperature is lower than the first temperature. The second temperature is a temperature suitable for an annealing reaction and an elongation reaction, and is, for example, 55° C. or higher and 75° C. or lower. That is, in this step, in the heating for the annealing reaction for the primer, the elongation reaction is performed. That is, the annealing reaction and the elongation reaction are performed at the same temperature. According to the above-mentioned FIG. 1, from the viewpoint of the activity efficiency of the polymerase, as the second temperature, around 70° C. is most suitable. The type of the liquid 4 is not particularly limited as long as it can be heated to the second temperature by the second hot plate 12, and for example, an aqueous sodium chloride solution and an oil can be exemplified.

The arm 30 is configured such that one end 30a is fixed by the fixing section 32 and the other end 30b is a free end. The end 30b of the arm 30 supports the container 8 containing the reaction solution 6. The arm 30 is operated by a motor (not shown) such that the end 30b reciprocates arcuately while fixing the end 30a.

By the reciprocation of the arm 30, the reaction solution 6 is alternately placed in the liquid 2 heated to the first temperature and in the liquid 4 heated to the second temperature. According to this, thermal cycling for PCR can be performed for the reaction solution 6. The number of cycles of the thermal cycling in this step can be appropriately set by driving and stopping of the motor, and for example, 20 or more and 60 or less. The conveying time of the reaction solution 6 from the liquid 2 to the liquid 4 and the conveying time of the reaction solution 6 from the liquid 4 to the liquid 2 are, for example, about 0.5 seconds.

In the thermal cycling step (Step S2), a heating time for the denaturation reaction per cycle (in the example shown in the drawing, a time in which the reaction solution 6 is placed in the liquid 2) is, for example, 0.3 seconds or more and 5 seconds or less, preferably 0.5 seconds or more and 2 seconds or less. By setting the heating time for the denaturation reaction to 0.3 seconds or more, it is possible to suppress insufficient denaturation due to a too short denaturation reaction time. By setting the heating time for the denaturation reaction to 5 seconds or less, the PCR speed can be increased.

In the thermal cycling step (Step S2), a heating time for the annealing reaction and the elongation reaction per cycle (in the example shown in the drawing, a time in which the reaction solution 6 is placed in the liquid 4) is, for example, 6 seconds or less, preferably 4 seconds or less, more preferably 3 seconds or less, furthermore preferably 1 second or more and 1.5 seconds or less. By setting the heating time for the annealing reaction and the elongation reaction to 6 seconds or less, the PCR speed can be increased.

In the thermal cycling step (Step S2), a time per cycle of the thermal cycling is 9 seconds or less, preferably 7 seconds or less, more preferably 6 seconds or less. By setting the time per cycle to 9 seconds or less, the thermal cycling speed can be increased. The time per cycle of the thermal cycling includes a time required for the denaturation reaction, the annealing reaction, and the elongation reaction, and the conveying time of the reaction solution for performing these reactions (for example, the conveying time of the reaction solution 6 from the liquid 2 to the liquid 4 and the conveying time of the reaction solution 6 from the liquid 4 to the liquid 2).

In the thermal cycling step (Step S2), a temperature decreasing rate from a high temperature to a low temperature and a temperature increasing rate from a low temperature to a high temperature of the reaction solution is, for example, 8° C./sec or more and 11° C./sec or less, preferably 9° C./sec or more and 10° C./sec or less, more preferably 9.2° C./sec or more and 9.6° C./sec or less.

In the thermal cycling step (Step S2), the number of bases of a nucleic acid to be amplified may be 200 or less. According to this, the PCR speed can be increased.

Subsequently, the fluorescence intensity of the reaction solution is measured (Step S3). For example, the reaction solution after thermal cycling is performed is transferred to a light transmissive container, and the fluorescence intensity is measured by irradiating the light transmissive container with light. By doing this, the amplification amount of the nucleic acid can be quantitatively determined.

### 3. EXPERIMENTAL EXAMPLES

Hereinafter, the invention will be more specifically described by showing experimental examples. However, the invention is by no means limited to the following experimental examples.

### 3.1. First Experimental Example

### 3.1.1. Preparation of Reaction Solution and Experimental Method

### (1) Example

As a template nucleic acid (template DNA), a Mycoplasma species DNA was used. The following reaction solution was prepared by adding this template nucleic acid to a nucleic acid amplification reaction reagent.

### Composition of Reaction Solution

| | | |
|---|---|---|
| Platinum Taq polymerase (5 units/μL) | 0.4 | μL |
| Buffer | 2.0 | μL |
| dNTP (10 mM) | 0.25 | μL |
| Forward primer for detection of Mycoplasma species | 0.32 | μL |
| (100 μM) | | |
| Reverse primer for detection of Mycoplasma species | 0.32 | μL |
| (100 μM) | | |
| Fluorescently labeled probe for detection of Mycoplasma | 0.9 | μL |
| species (10 μM) | | |
| Mycoplasma species DNA (100 copies/μL) | 1.0 | μL |
| Distilled water | 4.81 | μL |

As the fluorescently labeled probe, TaqMan (registered trademark) probe manufactured by Sigma-Aldrich Co. LLC. was used.

The buffer (buffer solution) contains MgCl₂, Tris-HCl (pH 9.0), and KCl. The concentration of MgCl₂ contained in the reaction solution was set to 5 mM.

The Tm values and the sequences of the primers are as shown in the following Table 1.

TABLE 1

| | Tm (° C.) | SEQ ID NO: | Sequence |
|---|---|---|---|
| Forward primer | 77.72 | 1 | 5′ GGT GAA ATC CAG GTA CGG GTG AAG |
| | | | ACA CC 3′ |
| Reverse primer | 77.22 | 2 | 5′ GTC CTG ATC AAT ATT AAG CTA CAG TAA |
| | | | AGC TTG ACG GGG 3′ |

Five types of probes having a different Tm value were prepared. The Tm values and the sequences of the probes are as shown in the following Table 2. In Table 2, in the sequences, an artificial nucleic acid is underlined, and the type of the artificial nucleic acid is shown. The Tm values were measured by a method described in the below-mentioned “3.1.3. Measurement of Actually Measured Tm Value”.

TABLE 2

| | Modification | Tm (° C.) | SEQ ID NO: | Sequence |
|---|---|---|---|---|
| No. 1 | — | 67.35 | 3 | 5′ FAM-CGG GAC GGA AAG ACC-BHQ1 3′ |
| No. 2 | N-Me | 72.76 | 3 | 5′ FAM-CGG GAC GGA AAG ACC-BHQ1 3′ |
| No. 3 | LNA | 75.25 | 3 | 5′ FAM-CGG GAC GGA AAG ACC-BHQ1 3′ |
| No. 4 | MGB | 76.52 | 3 | 5′ FAM-CGG GAC GGA AAG ACC-NFQ-MGB 3′ |
| No. 5 | — | 78.75 | 4 | 5′ FAM-CGT TAG GCG CAA CGG GAC GGAAAG |
| | | | | ACC-BHQ1 3′ |

10 μL of the reaction solution as described above was placed in a container (Light Cycler Capillaries (20 μL) manufactured by Roche), and PCR was performed by allowing the container to reciprocate between a high-temperature region (90° C.) and a low-temperature region (66° C.) using the device as shown in FIG. 3. The number of cycles of the thermal cycling was set to 40. Thereafter, the reaction solution was transferred to a different container (MicroAmp Fast Reaction Tubes, manufactured by Applied Biosystems, Inc.), and a fluorescence intensity (endpoint fluorescence intensity) was measured using a Step one Plus Real-time PCR system manufactured by Applied Biosystems, Inc.

In the first experimental example, the PCR condition was set as shown in the following Table 3.

TABLE 3

| | | | | Conveying | |
|---|---|---|---|---|---|
| | Hot | Low | High | time between | |
| | start | temperature | temperature | water tanks | Total |
| | (sec) | (sec) | (sec) | (sec) | (sec) |
| Condition 1 | 10 | 2 | 2 | 0.5 | 210 |
| Condition 2 | 10 | 1 | 1 | 0.5 | 130 |

In Table 3, the “hot start” refers to a procedure in which in order to activate the polymerase, the reaction solution is initially heated to the high temperature (90° C.) The temperature decreasing rate from the high temperature to the low temperature and the temperature increasing rate from the low temperature to the high temperature of the reaction solution was set to 9.2° C./sec in the case of the condition 1 (high temperature: 2 sec/low temperature: 2 sec), and 9.6° C./sec in the case of the condition 2 (high temperature: 1 sec/low temperature: 1 sec).

### (2) Comparative Example

As a comparative example, the following reaction solution was prepared.

### Composition of Reaction Solution

| | | |
|---|---|---|
| Platinum Taq polymerase (5 units/μL) | 0.4 | μL |
| 10× Gene TaqNT buffer | 1.0 | μL |
| dNTP (10 mM) | 0.25 | μL |
| Forward primer for detection of Mycoplasma species (20 μM) | 0.4 | μL |
| Reverse primer for detection of Mycoplasma species (20 μM) | 0.4 | μL |
| Fluorescently labeled probe for detection of Mycoplasma | 0.2 | μL |
| species (10 μM) | | |
| Mycoplasma species DNA (100 copies/μL) | 1.0 | μL |
| Distilled water | 6.35 | μL |

The Tm values and the sequences of the primers are as shown in the following Table 4. As the probe, five types probes having a different Tm value were prepared in the same manner as in Table 2.

TABLE 4

| | Tm (° C.) | SEQ ID NO: | Sequence |
|---|---|---|---|
| Forward primer | 71.98 | 5 | 5′ AAA TCC AGG TAC GGG TGA AG 3′ |
| Reverse primer | 70.48 | 6 | 5′ GTC CTG ATC AAT ATT AAG CTA CAG TAA A 3′ |

For 10 μL of the reaction solution as described above, PCR was performed for 40 cycles under the following condition: hot start: 2 min, high temperature (95° C.): 5 sec, low temperature (60° C.): 20 sec using a Step one Plus Real-time PCR system, and a fluorescence intensity (endpoint fluorescence intensity) was measured. The temperature decreasing rate and the temperature increasing rate of the reaction solution was set to 2.3° C./sec.

The Tm values shown in Tables 1, 2, and 4 are values actually measured using a Step one Plus Real-time PCR system. A given fluorescent substance is bound to a double-stranded DNA formed by a primer and a complementary strand thereto, and a decrease in the emission intensity from the fluorescent substance due to thermal denaturation is plotted against the temperature. A temperature at which a negative primary differential value of this graph reached a peak was defined as the Tm value.

### 3.1.2. Results of Measurement of Fluorescence Intensity

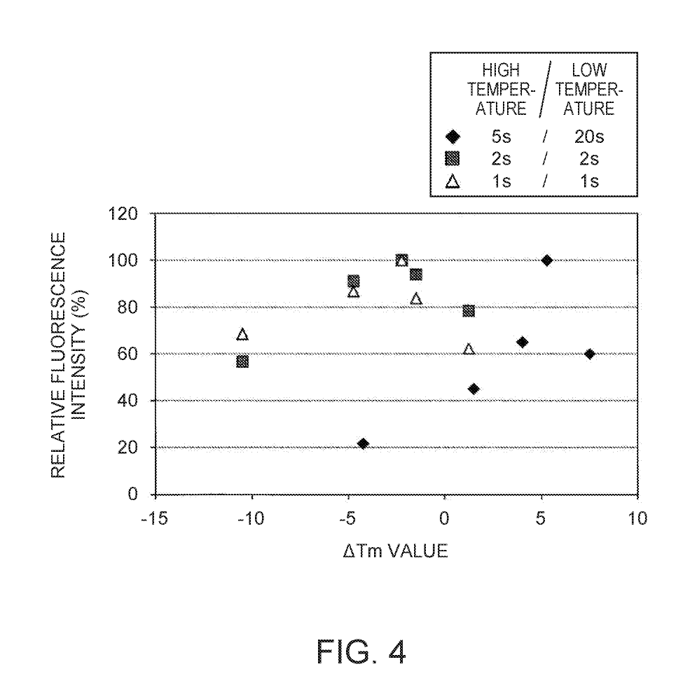

The results of measurement of the fluorescence intensity with respect to the above-mentioned examples (high temperature: 2 sec/low temperature: 2 sec, high temperature: 1 sec/low temperature: 1 sec) and comparative example (high temperature: 5 sec/low temperature: 20 sec) are shown. FIG. 4 is a graph showing a relationship between a value (ΔTm value) obtained by subtracting the Tm value of the primer from the Tm value of the probe and a relative fluorescence intensity. The ΔTm value is a value obtained by subtracting the average of the Tm value of the forward primer and the Tm value of the reverse primer from the Tm value of the probe.

The “relative fluorescence intensity” represented by the vertical axis in FIG. 4 is a relative intensity when the fluorescence intensity of the unreacted solution for which thermal cycling (temperature cycling) is not performed (background) is subtracted from the endpoint fluorescence intensity, and the highest intensity value at the measurement point was assumed to be 100%.

As shown in FIG. 4, in the case of the high temperature: 5 sec/low temperature: 20 sec, a relative fluorescence intensity resulted in 60% or more when the ΔTm value was in the range of 4.02° C. or more and 7.52° C. or less.

On the other hand, a relative fluorescence intensity resulted in 60% or more when the ΔTm value was in the range of −4.71° C. or more and 1.28° C. or less in the case of the high temperature: 2 sec/low temperature: 2 sec, and when the ΔTm value was in the range of −10.47° C. or more and 1.28° C. or less in the case of the high temperature: 1 sec/low temperature: 1 sec. Further, a relative fluorescence intensity resulted in 70% or more when the ΔTm value was in the range of −4.71° C. or more and 1.28° C. or less in the case of the high temperature: 2 sec/low temperature: 2 sec, and when the ΔTm value was in the range of −4.71° C. or more and −1.48° C. or less in the case of the high temperature: 1 sec/low temperature: 1 sec.

Therefore, it was found that in the case where the PCR speed is increased (the reaction time is reduced), by setting the ΔTm value to −11 or more and 2 or less, preferably −5 or more and 2 or less, the fluorescence intensity from the probe can be increased.

It is surprising that the fluorescence intensity is increased when the Tm value of the probe is lower than the Tm value of the primer. It is because as described above, when annealing of a primer occurs prior to hybridization of a probe, due to an elongation reaction by a polymerase, the probe cannot hybridize. In the case where the PCR speed is not increased (in the case of the high temperature: 5 sec/low temperature: 20 sec), in FIG. 4, when the Tm value of the probe is lower than the Tm value of the primer (when the ΔTm value is a negative value), the relative fluorescence intensity is only about 20%. On the other hand, in the case where the PCR speed is increased (in the case of the high temperature: 2 sec/low temperature: 2 sec and in the case of the high temperature: 1 sec/low temperature: 1 sec), when the ΔTm value is rather a negative value, the fluorescence intensity is high.

The cause for such a phenomenon is considered, for example, as follows. In the case of high-speed PCR, the temperature decreasing rate is large, and even if the primer anneals, before elongation by the polymerase reaches the probe-binding region (the region to which the probe hybridizes), the temperature of the reaction solution is decreased to a temperature at which the probe can hybridizes. Due to this, the probe hybridizes before elongation by the polymerase reaches the probe-binding region, and is hydrolyzed. In the case where the PCR speed is not increased, (i) hybridization of the probe, (ii) annealing of the primer, (iii) priming of the polymerase, and (iv) elongation by the polymerase occur in this order. However, in the case where the PCR speed is increased, it is considered that the reaction proceeds in the following order: (ii), (iii), (i), and (iv). However, this presumption is merely a hypothesis, and an additional experiment is considered to be required for elucidation of the cause.

### 3.1.3. Measurement of Actually Measured Tm Value

The Tm value (actually measured Tm value) used in the first experimental example is an actually measured value determined according to the following method.

### Composition of Reaction Solution

| | |
|---|---|
| Probe or primer whose Tm value is desired to be measured | 5.0 μL |
| (10 μM) | |
| Complementary strand (100 μM) | 0.5 μL |
| SYBR Green (25 nM) | 0.2 μL |
| Buffer | 2.0 μL |
| Distilled water | 2.3 μL |

The “complementary strand” refers to a strand complementary to the “probe or primer whose Tm value is desired to be measured”. Further, the buffer contains MgCl₂, Tris-HCl (pH 9.0), and KCl. The concentration of MgCl₂ contained in the reaction solution was set to 5 mM.

The reaction solution was placed in a sample tube, and the Tm value was actually measured using a Step one Plus Real-time PCR system. Specifically, the reaction solution was heated to 99° C. for 2 minutes, subsequently heated to 45° C. for 1 minute, and thereafter heated to 99° C. for 15 seconds. The condition that the temperature was increased from 45° C. to 99° C. was 0.5° C./sec, and the fluorescence intensity was measured during this procedure. The fluorescence intensity and the temperature were graphed, and a temperature at which a negative primary differential value of this graph reached a peak was defined as the actually measured Tm value (actually measured Tm value).

### 3.2. Second Experimental Example

### 3.2.1. Preparation of Reaction Solution and Experimental Method

As a template nucleic acid (template DNA), a Mycoplasma species DNA was used. The following reaction solution was prepared by adding this template nucleic acid to a nucleic acid amplification reaction reagent.

### Composition of Reaction Solution

| | | |
|---|---|---|
| Platinum Taq polymerase (5 units/μL) | 0.4 | μL |
| Buffer | 2.0 | μL |
| dNTP (10 mM) | 0.25 | μL |
| Forward primer for detection of Mycoplasma species (20 μM) | 1.2 | μL |
| Reverse primer for detection of Mycoplasma species (20 μM) | 1.2 | μL |
| Fluorescently labeled probe for detection of Mycoplasma | 0.9 | μL |
| species (10 μM) | | |
| Mycoplasma species DNA (100 copies/μL) | 1.0 | μL |
| Distilled water | 3.05 | μL |

As the fluorescently labeled probe, TaqMan (registered trademark) probe manufactured by Sigma-Aldrich Co. LLC. was used.

The buffer (buffer solution) contains MgCl₂, Tris-HCl (pH 9.0), and KCl. The concentration of MgCl₂ contained in the reaction solution was set to 5 mM.

In this experiment, primers having a different Tm value were used. Specifically, primers having a Tm value of about 60° C. (Tm60), about 70° C. (Tm70), about 75° C. (Tm75), about 80° C. (Tm80), or about 85° C. (Tm85) were used. The Tm values and the sequences of the primers having a Tm value of about 60° C., about 70° C., about 75° C., about 80° C., or about 85° C., are as shown in the following Table 5. The Tm value and the sequence of the probe are the same as those of “No. 1” in Table 2.

TABLE 5

| | | Tm (° C.) | SEQ ID NO: | Sequence |
|---|---|---|---|---|
| Tm 60 | Forward | 62.6 | 7 | 5′ AAA TCC AGG TAC GGG TGA AG 3′ |
| | primer | | | |
| | Reverse | 60.6 | 8 | 5′ GTC CTG ATC AAT ATT AAG CTA CAG TAA A 3′ |
| | primer | | | |
| Tm 70 | Forward | 70.4 | 9 | 5′ AAA TCC AGG TAC GGG TGA AGA CAC C 3′ |
| | primer | | | |
| | Reverse | 70.7 | 10 | 5′ GTC CTG ATC AAT ATT AAG CTA CAG TAA AGC TTG |
| | primer | | | ACG 3′ |
| Tm 75 | Forward | 75.9 | 11 | 5′ GGT GAA ATC CAG GTA GGG GTG AAG ACA CC 3′ |
| | primer | | | |
| | Reverse | 75.4 | 12 | 5′ GTC CTG ATC AAT ATT AAG CTA CAG TAA AGC TTG |
| | primer | | | ACG GGG 3′ |
| Tm 80 | Forward | 80.2 | 13 | 5′ GGT GAA ATC CAG GTA GGG GTG AAG ACA CCC G 3′ |
| | primer | | | |
| | Reverse | 79.0 | 14 | 5′ CAT GAT AAT GTG CTG ATC AAT ATT AAG CTA CAG TAA |
| | primer | | | AGC TTG ACG GGG TC 3′ |
| Tm 85 | Forward | 85.5 | 15 | 5′ GGT GAA ATC CAG GTA GGG GTG AAG ACA CCC GTT |
| | primer | | | AGG GGG 3′ |
| | Reverse | | | 5′ GCA TCG ATT GCT CCT ACC TAT TCT CTA CAT GAT AAT |
| | primer | 84.9 | 16 | GTG CTG ATC AAT ATT AAG CTA CAG TAAAGC TTG ACG |
| | | | | GGG TC 3′ |

The Tm values shown in Table 5 were calculated according to the above-mentioned formula (1), and the calculation was performed by setting Ct to 500 nM and Na⁺ to 50 mM in the formula (1). As the probe, “No. 1” shown in Table 2 was used.

10 μL of the reaction solution as described above was placed in a container (Light Cycler Capillaries (20 μL) manufactured by Roche), and PCR was performed by allowing the container to reciprocate between a high-temperature region and a low-temperature region using the device as shown in FIG. 3. The number of cycles of the thermal cycling was set to 40. Thereafter, the reaction solution was transferred to a different container (MicroAmp Fast Reaction Tubes, manufactured by Applied Biosystems, Inc.), and a fluorescence intensity was measured using a Step one Plus Real-time PCR system manufactured by Applied Biosystems, Inc.

In the PCR using each of the primers (Tm60, Tm70, Tm75, Tm80, and Tm85), the heating temperature (high temperature) for the denaturation reaction was set to 87° C. In the PCR using Tm60, Tm70, Tm75, Tm80, and Tm85, the heating temperature (low temperature) for the annealing reaction and the elongation reaction was set to 60° C., 63° C., 66° C., 69° C., and 72° C., respectively. In the PCR, the heating time (the time at the high temperature) for the denaturation reaction per cycle, the heating time (the time at the low temperature) for the annealing reaction and the elongation reaction per cycle, and the reaction time are shown in the following Table 6. Incidentally, in order to activate the polymerase, the reaction solution was initially heated to the high temperature for 10 seconds (hot start). The reaction time is obtained by, in addition to the polymerase activation time, adding the time at the high temperature and the time at the low temperature multiplied by 40 (the number of cycles), and further adding the conveying time of the reaction solution. Further, in Table 6, the time per cycle of the thermal cycling is obtained by adding the conveying time from the high-temperature region to the low-temperature region (0.5 sec) and the conveying time from the low-temperature region to the high-temperature region (0.5 sec) to the sum of the time at the high temperature and the time at the low temperature. For example, in the case where the reaction time is 370 seconds, the time per cycle of the thermal cycling is as follows: the time at the high temperature (2 sec)+the time at the low temperature (6 sec)+the conveying time from the high-temperature region to the low-temperature region (0.5 sec)+the conveying time from the low-temperature region to the high-temperature region (0.5 sec)=9 sec.

TABLE 6

| | | | | | | | |
|---|---|---|---|---|---|---|---|
| Time at high temperature (sec) | 2 | 2 | 2 | 2 | 2 | 2 | 4 |
| Time at low temperature (sec) | 1 | 1.5 | 2 | 3 | 4 | 6 | 6 |
| Reaction time (sec) | 170 | 190 | 210 | 250 | 290 | 370 | 450 |

### 3.2.2. Results of Measurement of Fluorescence Intensity

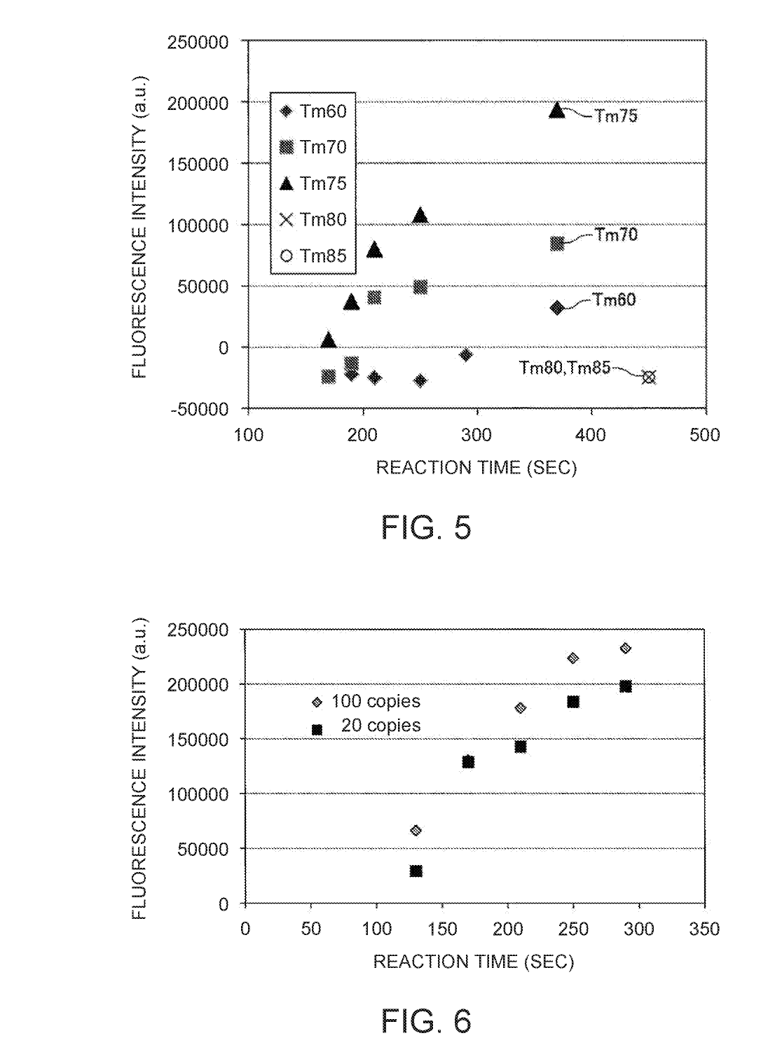

FIG. 5 is a graph showing a relationship between a PCR reaction time and a fluorescence intensity. As shown in FIG. 5, in the case where the time at the low temperature was 6 seconds (in the case where the time per cycle was 9 seconds), amplification was confirmed when using Tm70 and Tm75, however, in the case where the time at the low temperature was 4 seconds (in the case where the time per cycle was 7 seconds) or less, amplification of a nucleic acid was not confirmed when using Tm60, and amplification was confirmed when using Tm70 and Tm75. Therefore, it was found that by setting the Tm value of the primer to 70° C. or higher and lower than 80° C., even in the case of high-speed PCR in which the time at the low temperature is 4 seconds or less, a nucleic acid can be amplified. This is considered to be because a primer having a higher Tm value anneals to a template nucleic acid faster, and therefore, Tm70 and Tm75 are more suitable for increasing the thermal cycling speed than Tm60. Further, according to the above-mentioned FIG. 1, it is considered that Tm70 and Tm75 have a higher polymerase activity efficiency than Tm60 and can accelerate the elongation reaction, and therefore, Tm70 and Tm75 are more suitable for increasing the thermal cycling speed than Tm60. When considering a variation in the device used in this experimental example, it can be said that when the fluorescence intensity is 35000 or more, a nucleic acid is reliably amplified. Therefore, in the case where the time per cycle is 9 seconds, it cannot be said that a nucleic acid is reliably amplified when using Tm60, and it can be said that a nucleic acid is reliably amplified when using Tm70 and Tm75.

Further, from FIG. 5, it was found that by setting the time at the low temperature to 2 seconds or more and 4 seconds or less, and the Tm value of the primer to 70° C. or higher and 75° C. or lower, a nucleic acid can be more reliably amplified even if the time at the low temperature is 4 seconds or less. Further, in FIG. 5, when using Tm80 and Tm85, amplification of a nucleic acid was not confirmed. This is considered to be because the Tm value was too high, and therefore, a primer dimer or the like was formed.

In FIG. 5, a value obtained by subtracting the fluorescence intensity of the unreacted solution for which thermal cycling was not performed (background) from the endpoint fluorescence intensity is plotted. The plot in which the fluorescence intensity shows a negative value is considered to be a measurement error.

### 3.3. Third Experimental Example

### 3.3.1. Preparation of Reaction Solution and Experimental Method

As a template nucleic acid (template DNA), a Bordetella pertussis DNA was used. The following reaction solution was prepared by adding this template nucleic acid to a nucleic acid amplification reaction reagent.

### Composition of Reaction Solution

| | | |
|---|---|---|
| Platinum Taq polymerase (5 units/μL) | 0.4 | μL |
| Buffer | 2.0 | μL |
| dNTP (10 mM) | 0.25 | μL |
| Forward primer for detection of Bordetella pertussis (100 μM) | 0.32 | μL |
| Reverse primer for detection of Bordetella pertussis (100 μM) | 0.32 | μL |
| Fluorescently labeled probe for detection of Bordetella | 0.9 | μL |
| pertussis (10 μM) | | |
| Bordetella pertussis DNA (20 copies or 100 copies/μL) | 1.0 | μL |
| Distilled water | 4.81 | μL |

As the fluorescently labeled probe, TaqMan (registered trademark) probe manufactured by Sigma-Aldrich Co. LLC. was used.

The buffer (buffer solution) contains MgCl₂, Tris-HCl (pH 9.0), and KCl. The concentration of MgCl₂ contained in the reaction solution was set to 5 mM.

The Tm values and the sequences of the primers, and the sequence of the probe are as shown in the following Table 7. The Tm values shown in Table 7 were calculated in the same manner as the Tm values shown in Table 5.

TABLE 7

| | Tm (° C.) | SEQ ID NO: | Sequence |
|---|---|---|---|
| Forward | 80.8 | 17 | 5′ ATC AAG CAC CGC TTT ACC CGA CCT TAC CGC C |
| primer | | | 3′ |
| Reverse | 80.3 | 18 | 5′ TTG GGA GTT CTG GTA GGT GTG AGC GTA AGC |
| primer | | | CCA 3′ |
| Probe | | 19 | 5′ FAM-AAT GGC AAG GCC GAA CGC TTC |
| | | | A-NFQ-MGB 3′ |

PCR was performed for 10 μL of the reaction solution as described above by performing hot start for 10 seconds in the same manner as in the first experimental example. The high temperature was set to 90° C., and the low temperature was set to 60° C. The heating time (the time at the high temperature) for the denaturation reaction per cycle, the heating time (the time at the low temperature) for the annealing reaction and the elongation reaction per cycle, and the reaction time are shown in the following Table 8.

TABLE 8

| | | | | | |
|---|---|---|---|---|---|
| Time at high temperature (sec) | 1 | 2 | 2 | 2 | 2 |
| Time at low temperature (sec) | 1 | 1 | 2 | 3 | 4 |
| Reaction time (sec) | 130 | 170 | 210 | 250 | 290 |

### 3.3.2. Results of Measurement of Fluorescence Intensity

FIG. 6 is a graph showing a relationship between a PCR reaction time and a fluorescence intensity. As shown in FIG. 6, even if the Tm value of the primer was about 80° C., amplification of a nucleic acid could be confirmed.

The invention includes substantially the same configurations (for example, configurations having the same functions, methods, and results, or configurations having the same objects and effects) as the configurations described in the embodiments. Further, the invention includes configurations in which a part that is not essential in the configurations described in the embodiments is substituted. Further, the invention includes configurations having the same effects as in the configurations described in the embodiments, or configurations capable of achieving the same objects as in the configurations described in the embodiments. In addition, the invention includes configurations in which known techniques are added to the configurations described in the embodiments.

The entire disclosure of Japanese Patent Application No. 2016-149491, filed July29, 2016 is expressly incorporated by reference herein.

### Sequence Listing Free Text

SEQ ID NO: 1 is the sequence of a forward primer for Mycoplasma bacteria.

SEQ ID NO: 2 is the sequence of a reverse primer for Mycoplasma bacteria.

SEQ ID NO: 3 is the sequence of a fluorescently labeled probe for Mycoplasma bacteria.

SEQ ID NO: 4 is the sequence of a fluorescently labeled probe for Mycoplasma bacteria.

SEQ ID NO: 5 is the sequence of a forward primer for Mycoplasma bacteria.

SEQ ID NO: 6 is the sequence of a reverse primer for Mycoplasma bacteria.

SEQ ID NO: 7 is the sequence of a forward primer for Mycoplasma bacteria.

SEQ ID NO: 8 is the sequence of a reverse primer for Mycoplasma bacteria.

SEQ ID NO: 9 is the sequence of a forward primer for Mycoplasma bacteria.

SEQ ID NO: 10 is the sequence of a reverse primer for Mycoplasma bacteria.

SEQ ID NO: 11 is the sequence of a forward primer for Mycoplasma bacteria.

SEQ ID NO: 12 is the sequence of a reverse primer for Mycoplasma bacteria.

SEQ ID NO: 13 is the sequence of a forward primer for Mycoplasma bacteria.

SEQ ID NO: 14 is the sequence of a reverse primer for Mycoplasma bacteria.

SEQ ID NO: 15 is the sequence of a forward primer for Mycoplasma bacteria.

SEQ ID NO: 16 is the sequence of a reverse primer for Mycoplasma bacteria.

SEQ ID NO: 17 is the sequence of a forward primer for Bordetella pertussis.

SEQ ID NO: 18 is the sequence of a reverse primer for Bordetella pertussis.

SEQ ID NO: 19 is the sequence of a fluorescently labeled probe for Bordetella pertussis.

What is claimed is:

1. A nucleic acid amplification reaction method, comprising:
performing thermal cycling for amplifying a nucleic acid for a reaction solution containing a template nucleic acid, a primer, a probe, and a polymerase, wherein in the thermal cycling,
the time per cycle of the thermal cycling is 9 seconds or less,
the calculated Tm value of the primer is 65° C. or higher and 80° C. or lower,
a ΔTm value obtained by subtracting the actually measured Tm value of the primer from the actually measured Tm value of the probe is −11° C. or more and 2° C. or less,
the calculated Tm value is a value calculated according to the following formula (1), and
the actually measured Tm value is an actually measured value obtained by actual measurement:
Tm=1000 ΔH/(−10.8+ΔS+R×ln(Ct/4))−273.15+16.6 log [Na⁺] (1)
wherein ΔH represents the sum (kcal/mol) of the nearest neighbor enthalpy changes for hybrids, ΔS represents the sum (cal/mol/K) of the nearest neighbor entropy changes for hybrids, R represents the gas constant (1.987 cal/deg/mol), Ct represents the molar concentration (mol/L) of the primer, and Na⁺ represents the concentration (mol/L) of a monovalent cation contained in the buffer.

2. The nucleic acid amplification reaction method according to claim 1, wherein a heating time for an annealing reaction for the primer is 6 seconds or less.

3. The nucleic acid amplification reaction method according to claim 1, wherein the probe contains an artificial nucleic acid.

4. The nucleic acid amplification reaction method according to claim 1, wherein the probe contains a minor groove binder molecule.

5. The nucleic acid amplification reaction method according to claim 1, wherein the ΔTm value is −5° C. or more and 2° C. or less.

6. The nucleic acid amplification reaction method according to claim 1, wherein
the reaction solution contains a divalent cation, and
the concentration of the divalent cation contained in the reaction solution is 2 mM or more and 7.5 mM or less.

7. The nucleic acid amplification reaction method according to claim 1, wherein
the reaction solution contains MgCl₂,
the divalent cation is derived from MgCl₂, and
the concentration of MgCl₂ contained in the reaction solution is 4 mM or more and 7.5 mM or less.

8. The nucleic acid amplification reaction method according to claim 1, wherein
the reaction solution contains MgSO₄,
the divalent cation is derived from MgSO₄, and
the concentration of MgSO₄ contained in the reaction solution is 2 mM or more and 3 mM or less.

9. A nucleic acid amplification reaction reagent, which is a nucleic acid amplification reaction reagent for amplifying a nucleic acid, comprising a primer, a probe, a polymerase, and MgCl₂, wherein
when the nucleic acid amplification reaction reagent becomes a reaction solution for performing a nucleic acid amplification reaction, the concentration of MgCl₂ contained in the reaction solution is 4 mM or more and 7.5 mM or less,
the calculated Tm value of the primer is 65° C. or higher and 80° C. or lower,
a ΔTm value obtained by subtracting the actually measured Tm value of the primer from the actually measured Tm value of the probe is −11° C. or more and 2° C. or less,
the calculated Tm value is a value calculated according to the following formula (1), and
the actually measured Tm value is an actually measured value obtained by actual measurement:
Tm=1000 ΔH/(−10.8+ΔS+R×ln(Ct/4))−273.15+16.6 log [Na⁺] (1)
wherein ΔH represents the sum (kcal/mol) of the nearest neighbor enthalpy changes for hybrids, ΔS represents the sum (cal/mol/K) of the nearest neighbor entropy changes for hybrids, R represents the gas constant (1.987 cal/deg/mol), Ct represents the molar concentration (mol/L) of the primer, and Na⁺ represents the concentration (mol/L) of a monovalent cation contained in the buffer.

10. A nucleic acid amplification reaction reagent, which is a nucleic acid amplification reaction reagent for amplifying a nucleic acid, comprising a primer, a probe, a polymerase, and MgSO₄, wherein
when the nucleic acid amplification reaction reagent becomes a reaction solution for performing a nucleic acid amplification reaction, the concentration of MgSO₄ contained in the reaction solution is 2 mM or more and 3 mM or less,
the calculated Tm value of the primer is 65° C. or higher and 80° C. or lower,
a ΔTm value obtained by subtracting the actually measured Tm value of the primer from the actually measured Tm value of the probe is −11° C. or more and 2° C. or less,
the calculated Tm value is a value calculated according to the following formula (1), and
the actually measured Tm value is an actually measured value obtained by actual measurement:
Tm=1000 ΔH/(−10.8+ΔS+R×ln(Ct/4))−273.15+16.6 log [Na⁺] (1)
wherein ΔH represents the sum (kcal/mol) of the nearest neighbor enthalpy changes for hybrids, ΔS represents the sum (cal/mol/K) of the nearest neighbor entropy changes for hybrids, R represents the gas constant (1.987 cal/deg/mol), Ct represents the molar concentration (mol/L) of the primer, and Na⁺ represents the concentration (mol/L) of a monovalent cation contained in the buffer.

11. A method of using the nucleic acid amplification reaction reagent according to claim 9, comprising:
preparing the reaction solution by bringing the nucleic acid amplification reaction reagent and a template nucleic acid solution containing a template nucleic acid into contact with each other; and
amplifying a nucleic acid by performing thermal cycling in which the time per cycle is 9 seconds or less for the reaction solution.

12. A method of using the nucleic acid amplification reaction reagent according to claim 10, comprising:
preparing the reaction solution by bringing the nucleic acid amplification reaction reagent and a template nucleic acid solution containing a template nucleic acid into contact with each other; and
amplifying a nucleic acid by performing thermal cycling in which the time per cycle is 9 seconds or less for the reaction solution.
